(19) United States
(12) Patent Application Publication
(10) Pub. No.: US 2004/0132984 A1
(43) Pub. Date: Jul. 8, 2004

(54) Antisense compounds, methods and compositions for treating NGAL-related inflammatory disorders

(76) Inventors: Andreas Dieckmann, Bromma (SE); Robert Lofberg, Djursholm (SE); Oliver Von Stein, Spanga (SE); Petra Von Stein, Spanga (SE); Liam Good, Stockholm (SE)

Correspondence Address: YOUNG & THOMPSON, 745 SOUTH 23RD STREET 2ND FLOOR, ARLINGTON, VA, 22202

(21) Appl. No.: 10/620,576

(22) Filed: Jul. 17, 2003

Related U.S. Application Data
(60) Provisional application No. 60/407,954, filed on Sep. 5, 2002.

(30) Foreign Application Priority Data
Jul. 17, 2002 (SE) 0202244-0

(51) Int. Cl. A61K 48/00; C07H 21/04

(52) U.S. Cl. 536/23.2; 514/44

(57) ABSTRACT

The present invention relates antisense oligonucleotide compounds for use in modulating the function of nucleic acid molecules encoding neutrophil gelatinase associated lipocalin (NGAL). More specifically, the invention provides compounds of 8 to 50 nucleobases in length capable of specifically hybridising with nucleic acid molecules encoding NGAL and thereby inhibiting the expression of the NGAL protein product, as well as pharmaceutical compositions thereof and methods of their use.

### FIELD OF INVENTION

The present invention relates to the use of antisense oligonucleotides compounds as specific inhibitors of human lipocalin 2 (LCN2) also know as human neutrophil lipocalin or neutrophil gelatinase associated lipocalin (herein known as “NGAL”). The present invention further relates to methods and compositions for treating inflammatory disorders, comprising administering to the patient a therapeutic effective amount of said compounds.

### BACKGROUND OF THE INVENTION

The lipocalins are a family of extracellular ligand-binding proteins whose function among others is to bind and transport small hydrophobic molecules. Lipocalins function in a wide variety of processes including nutrient transport, cell growth regulation, immune response, inflammation and prostaglandin synthesis.

Members of the lipocalin family display unusually low levels of overall sequence conservation, however, each lipocalin associates with a particular ligand and delivers that ligand to appropriate target sites within the organism. Retinol-binding protein (RBP), one of the best characterized lipocalins, transports retinol from stores within the liver to target tissues. Apolipoprotein D (apo D), a component of high-density lipoproteins (HDLs) and low-density lipoproteins (LDLs), functions in the targeted collection and delivery of cholesterol throughout the body.

Lipocalins are also involved in cell regulatory processes and moreover, serve as diagnostic and prognostic markers in a variety of disease states. For example, the plasma level of alpha glycoprotein (AGP) is monitored during pregnancy and in diagnosis and prognosis of conditions including cancer chemotherapy, renal dysfunction, myocardial infarction, arthritis, and multiple sclerosis.

NGAL was first described in 1993 by Kjeldsen et al., who identified a novel liopcalin gene from human neutrophils. Neutrophil gelatinase-associated lipocalin (NGAL) is a 25-kDa lipocalin and exists in monomeric and homo- and heterodimeric forms, the latter as a dimer with human neutrophil gelatinase. It is secreted from specific granules of activated human neutrophils. Homologous proteins have been identified in mouse (24p3/uterocalin) and rat (alpha(2)-microglobulin-related protein/neu-related lipocalin). Structural data have confirmed a typical lipocalin fold of NGAL with an eight-stranded beta-barrel, but with an unusually large cavity lined with more polar and positively charged amino acid residues than normally seen in lipocalins. Chemotactic formyl-peptides from bacteria have been proposed as ligands of NGAL, but binding experiments and the structure of NGAL do not support this hypothesis. Besides neutrophils, NGAL is expressed in most tissues normally exposed to microorganisms, and its synthesis is induced in epithelial cells during inflammation (Carlson et al., 2002). This may indicate either a microbicidal activity of NGAL or a role in regulation of inflammation or cellular growth, however its function with respect to inflammation and cellular growth has yet to be identified.

In inflammatory and neoplastic disorders of the colon a defect barrier function of the mucosa may result in absorption of bacterial products from the intestinal lumen. These products may further recruit inflammatory cells and thus augment the inflammatory response. NGAL is capable of binding bacterial formylpeptides, and a very high expression of NGAL was demonstrated in colonic epithelium in areas of inflammation, both in non-malignant epithelium (diverticulitis, inflammatory bowel disease, and appendicitis) as well as in premalignant and malignant neoplastic lesions of the colon (Nielsen et al., 1996). The authors concluded that NGAL may serve as an important anti-inflammatory function as a scavenger of bacterial products. Moreover, Mallbris et al., 2002 demonstrated that NGAL is strongly induced in various disease conditions of the epidermis such as psoriasis, pityriasis rubra and squamous cell carcinoma.

Interestingly, NGAL has been shown to provide protective function of certain metalloprotinases (MMPs), such as MMP-9. NGAL is capable of protecting MMP-9 from degradation in a dose-dependent manner and thereby preserving MMP-9 enzymatic activity (Yan et al., 2001; Tschesche et al., 2001). In this manner, NGAL appears to potentiate the action of MMPs secreted from example pro-inflammatory type cells such as macrophages. In view of these results, NGAL can exert an enzyme-activating effect in the regulation of inflammatory and pathophysiological responses of pro-inflammatory type cells in the physiological activation of MMPs.

MMPs are required by macrophages to enable migration of macrophages into sites of inflammation (Shipley et al., 1996), furthermore, macrophages themselves are instrumental in the maintenance of inflammation, in that they are responsible for the production of many pro-inflammatory cytokines.

NGAL has also been described in connection with subclinical pulmonary emphysema (Betsuyaku et al., 1999), as well as being over expressed in the epidermis in a variety of skin disorders characterized by dysregulated epithelial differentiation such as psoriasis, pityriasis rubra and squamous cell carcinoma (Mallbris et al., 2002). Also of interest were the findings made by Eichler et al., in 1999, that patients suffering from cystic fibrosis, demonstrated elevated levels of NGAL in blood.

In light of the above, it appears that NGAL is involved in a number of inflammatory diseases and that by selectively inhibiting protein production by antisense modulation the possibility for new anti-inflammatory therapies to treat such disorders opens. There are very few selective inhibitors of NGAL reported and no selective inhibitor has been approved or marketed for the treatment of any disease in any mammal.

### SUMMARY OF THE INVENTION

The present invention provides antisense oligonucleotide compounds for use in modulating the function of nucleic acid molecules encoding human NGAL, ultimately by modulating the amount of NGAL produced. More specifically, the invention provides compounds of 8 to 50 nucleobases in length capable of specifically hybridising with nucleic acid molecules encoding NGAL and thereby blocking the production of the NGAL protein product. Further provided are methods and compositions of modulating the expression of NGAL in cells or tissues comprising contacting said cells or tissues with one or more of the antisense compounds or compositions of the invention. This is achieved by providing antisense compounds which specifically hybridise with nucleic acids encoding the NGAL protein product.

The invention is further defined in the attached claims, incorporated herein by reference.

### DESCRIPTION OF FIGURES

The invention will be described in closer detail in the following description, examples, and attached drawings, in which

FIG. 1(A) and (B) show RT-PCR analysis of the expression levels of NGAL using NGAL specific primers on mRNA derived from biopsy samples from 8 ulcerative colitis (UC) patients and 8 Crohn's disease (CD) patients respectively. (Key: M is a base-pair marker; C represents a biopsy sample taken from a non inflamed area; and T represents a biopsy taken from an inflamed area from the same patient. Numbers in brackets indicates patient number and the horizontal bar denotes a C and T biopsy sample derived from the same patient).

Alpha actin is used as a loading control and indicates the expression status of a housekeeping gene used commonly to demonstrate equal mRNA input in all RT-PCR reactions.

FIG. 2 shows the histogram of different criteria used to assess improvement in the degree of inflammation of the gastrointestinal tract after administration of an antisense compound. In this example, the antisense compound is that given by SEQ.ID.NO 3 and the experimental protocol is given by example 7.

(Key: The black solid bar denotes healthy animals that received only standard drinking water (healthy control). The hatched bar denotes colitis induced animals who receive 2.5% DSS in their drinking water which will induce inflammation of the colon (sick control). The chequered bar denotes those animals who received in addition to DSS in their drinking water, antisense compound SEQ.ID.NO 3 as outlined in example 2). Thick black bars denote negative control vs. sick animal control. Thin black bars denote comparison of sick animal control vs. NGAL antisense treated group. Histology was graded 0-4 according to the scale shown in Table 1. Significance is indicated as *P<0.05, **P<0.001 and ***P<0.0005. Error bars: SEM.

### DETAILED DESCRIPTION OF THE INVENTION

Before the present method is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

In the context of the invention, “antisense” as in “antisense molecules” and “antisense sequences” refers to single stranded RNA or DNA molecules complementary to a portion of the mRNA of a target gene. The antisense molecules will base pair with the mRNA, thus preventing translation of the mRNA into protein. Consequently, the term “antisense therapy” refers to methods using such antisense compounds which specifically hybridise to a target nucleic acid and modulate its function or translation, for example by suppressing or reducing the expression of gene products coded by said sequence.

In the context of the present invention, “complementary” refers to the capacity for precise pairing between two nucleotides.

Within the context of the present invention, “hybridization” refers to hydrogen bonding, which may be Watson-Crick, Hoogsteen or reverse Hoogsteen hydrogen bonding, between complementary nucleoside or nucleotide bases. Thus complementarity and hybridisation are terms used to indicate a sufficient degree of complementarity or precise paring such that stable and specific binding occurs between the oligonucleotide and the DNA or RNA target.

An antisense compound is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-specific target sequences under conditions in which specific binding is desired.

Furthermore, in the context of the present invention, “hybridisation under stringent conditions” refers to the criteria regarding temperature and buffers well know to those skilled in the art (Ausubel et al., 1991).

As is well known in the art, “functionally homologous” means sequences sharing perhaps a lower structural homology with the disclosed sequence, but exhibiting homologous function in vivo, in either the healthy or the diseased organism, e.g. coding the same or highly similar proteins with similar cellular functions.

As is well known in the art, “functionally inserted” or “operationally inserted” denotes that a sequence has been inserted in a host genome in such orientation, location and with such promoters and/or enhancers, where applicable, that the correct expression of said sequence occurs.

In the context of the present invention, “modulation” means either an increase (stimulation) or a decrease (inhibition) in the expression of a gene. In the context of the present invention, inhibition is the preferred form of modulation of gene expression and mRNA is the preferred target.

The present invention provides oligonucleotide compounds for use in modulating the function of nucleic acid molecules encoding a mammalian neutrophil gelatinase associated lipocalin (NGAL), ultimately by modulating the amount of NGAL produced. More specifically, said compound is an antisense oligonucleotide complementary to the mRNA of the NGAL. The modulation is achieved by providing antisense compounds, which specifically hybridise with nucleic acids encoding the NGAL protein product and thereby inhibit the translation of the NGAL. In one embodiment the target sequence is human and the antisense compound preferably hybridises to SEQ ID NO. 1 (GenBank® Acc. No. BC033089) or equivalent functional homologues thereof.

The antisense compounds in accordance with this invention preferably comprise from about 8 to about 50 nucleobases in length. Antisense oligonucleotides comprising from about 8 to about 30 nucleobases (i.e. from about 8 to about 30 linked nucleosides in length) are particularly preferred, and oligonucleotides comprising about 16 to 24 nucleobases are most preferred.

The invention also relates to a compound 8 to 50 nucleobases in length targeted to a nucleic acid molecule encoding mouse 24p3/uterocalin, wherein said compound specifically hybridises to and inhibits the translation of 24p3/uterocalin. In one embodiment the target sequence is SEQ ID NO. 2 (GenBank® Acc. No. XM130171) or an equivalent functional homologue thereof. These compounds are preferably oligonucleotides comprising from about 8 to about 30 nucleobases (i.e. from about 8 to about 30 linked nucleosides in length), most preferably about 16 to 24 nucleobases.

The antisense oligonucleotide according to the invention is either a DNA molecule or a RNA molecule. The invention makes available nucleic acid molecules in the form of antisense oligonucleotide molecules as defined above, and in particular SEQ ID NO. 3-11 (See Table 2, and the attached Sequence Listing, prepared using PatentIn 3.1), capable of specifically hybridising with nucleic acid molecules encoding NGAL and thereby blocking the production of the NGAL protein product.

In yet a further context of this invention, the term “oligonucleotide” refers to an oligomer or polymer of ribonucleic acid (RNA) or deoxyribonucleic acid (DNA) or mimetics thereof. This term also covers those oligonucleotides composed of naturally occurring nucleobases, sugars and covalent internucleoside (backbone) linkages as well as oligonucleotides having non-naturally-occurring modifications. As is known in the art, the phosphate groups within the oligonucleotide structure are commonly referred to as forming the internucleoside backbone of the oligonucleotide. The natural linkage or backbone of RNA and DNA is a 3′ to 5′ phosphodiester linkage.

Specific examples of preferred antisense compounds useful in this invention include oligonucleotides containing modified backbones or non-natural internucleoside linkages. These modifications have allowed one to introduce certain desirable properties that are not offered through naturally occurring oligonucleotides, such as reduced toxic properties, increased stability against nuclease degradation and enhanced cellular up-take.

According to a further embodiment, said antisense oligonucleotide comprises at least one modified nucleobase, which may be chemically modified by substitution in a non-bridging oxygen atom of the antisense nucleic acid backbone with a moiety selected from the group consisting of methane phosphate, methyl phosphate and phosphorothioate. phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates and thionoalkylphosphotriesters.

According to one embodiment, said substitution may take place at one or more nucleotides independently selected from the final three nucleotides at the 3′-terminus and/or 5′-terminus of said oligonucleotide. It is also conceived, that the substitution can occur at any position along the entire length of said oligonucleotide, or indeed all intranucleoside linkages are subjected to modification. Preferably, said oligonucleotide comprises at least one modified sugar moiety nucleobase, and the modified sugar moiety may be a 2′-O-methoxyethyl sugar moiety.

Said antisense agent may also be an antisense agent composed of DNA or RNA or an analogue or mimic of DNA or RNA including but not restricted to the following: methylphosphonate, N3′->P5′-phosphoramidate, morpholino, peptide nucleic acid (PNA), locked nucleic acid (LNA), arabinosyl nucleic acid (ANA), fluoro-arabinosyl nucleic acid (FANA) methoxy-ethyl nucleic acid (MOE). Preferably, said antisense agent is a homo or heteropolymer containing combinations of the above DNA or RNA or analogues or mimics of DNA or RNA.

In a further embodiment, the antisense compounds of the present invention can be utilized for therapeutics and as prophylaxis. For therapeutics, an animal, preferably a human, suspected of having a disease or disorder, which can be treated by modulating the expression of NGAL, is treated by administering a therapeutically effective amount of antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically (i.e. to delay the onset of a disease or condition in which NGAL is suspected of being involved).

In yet another embodiment, the antisense compounds of the invention are useful for research and diagnostics of human subjects, because these compounds hybridize to nucleic acids encoding NGAL, enabling sandwich and other assays to easily be designed to exploit this fact. Hybridization of the antisense oligonucleotides of the invention with a nucleic acid encoding NGAL and the resulting suppression/inhibition in expression of NGAL can be detected by means well known in the art. For example, radiolabeling of the antisense compound, RNase protection assays, will demonstrate specific hybridisation of the antisense compound to the target mRNA of NGAL. Various means of detecting reduced levels of NGAL can be employed well known in the art such as antibody detection of NGAL, or enzymatic based activity assays. In another embodiment the antisense compounds are used in a method of inhibition of the expression of NGAL in cells or tissues, wherein said cells or tissues are contacted in vivo or in vitro with a therapeutically effective dose of the compound or composition of the invention, thereby inhibiting the expression of NGAL. Preferably, said inhibition suppresses a NGAL dependent process in a human subject. The NGAL dependent process is most preferably one of inflammatory bowel disease, such as ulcerative colitis and Crohn's disease, rheumatoid arthritis, psoriasis, and asthma.

Another embodiment of the invention relates to a method of diagnosing inflammatory bowel disease in a human subject comprising screening for the presence or absence of the expression of NGAL and the expression of NGAL is an indication of inflammatory bowel disease.

The invention in particular provides compounds and methods for the treatment of an animal, particular a human suspected of having or being prone to a human disease associated with inappropriate modulation of NGAL, by administrating a therapeutic or prophylactically effective amount of one or more antisense compound or compositions of the invention designed to modulate expression of NGAL.

Because they are selective, the compounds of the present application are expected to be useful for long-term therapy with less of the complications related to known broad-spectrum inhibition. Thus, while the compounds of the present application are useful for the treatment of a variety of NGAL mediated diseases and conditions, these selective inhibitors are particularly useful for the treatment of disorders that have a significant inflammatory component. Targeting NGAL by antisense compounds may ultimately reduce the capacity of pro-inflammatory cells reaching sites of inflammation and thereby exert a potent anti-inflammatory effect. This could offer a new therapeutic possibility to treat inflammatory conditions.

As an alternative to targeted antisense delivery, targeted ribozymes may be used. The term “ribozyme” refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, make mixed RNA/DNA oligonucleotides, or modify the phosphodiester backbone.

Another alternative to antisense is the use of so-called “RNA interference” (RNAi). Double-stranded RNAs (dsRNAs) can provoke gene silencing in numerous in vivo contexts including Drosophila, Caenorhabditis elegans, planaria, hydra, in trypanosomes, fungi plants and mammals. The natural function of RNAi and co-suppression appears to be protection of the genome against invasion by mobile genetic elements such as retrotransposons and viruses which produce aberrant RNA or dsRNA in the host cell when they become active (Jensen et al., 1999; Ketting et al., 1999; Ratcliff et al., 1999; Tabara et al., 1999). Specific mRNA degradation prevents transposon and virus replication although some viruses are able to overcome or prevent this process by expressing proteins that suppress PTGS (Lucy et al., 2000). The double-stranded RNA molecule may be prepared by a method comprising the steps: (a) synthesizing two RNA strands each having a length from 19-25, e.g. from 19-23 nucleotides, wherein said RNA strands are capable of forming a double-stranded RNA molecule, wherein preferably at least one strand has a 3′-overhang from 1-5 nucleotides, (b) combining the synthesized RNA strands under conditions, wherein a double-stranded RNA molecule is formed, which is capable of media ting target-specific nucleic acid modifications, particularly RNA interference and/or DNA methylation. In one embodiment the antisense RNAi comprises at least an 8 nucleotide portion included in one of the sequences of SEQ ID NO 3-11 and has a total length of no more than 25 nucleotides.

The dsRNA is usually administered as a pharmaceutical composition. The administration may be carried out by known methods, wherein a nucleic acid is introduced into a desired target cell in vitro or in vivo. Commonly used gene transfer techniques include calcium phosphate, DEAE-dextran, electroporation, microinjection and viral methods. Such methods are taught in Current Protocols in Molecular Biology, Ausubel et al., 1993.

The present invention also makes available a pharmaceutical composition, wherein said composition comprises a compound or antisense agent as describe above, and a pharmaceutically acceptable formulation and composition, carrier or diluent. Said pharmaceutical composition preferably further comprises a colloidal dispersion system. The pharmaceutical composition of the present invention may be administered in a number of ways depending largely on whether a local, topical or systemic mode of administration is most appropriate for the condition be treated. These different modes of administration are for example topical (e.g., on the skin), local (including ophthalmic and to various mucous membranes such for example vaginal, nasal, and rectal delivery), oral or parenteral and pulmonary.

The preparation of such compositions and formulations is generally known to those skilled in the pharmaceutical and formulation arts and may be applied to the formulation of the composition of the present invention.

In the scope of this invention, preferred examples of pharmaceutically acceptable salts include but are not limited to (a) salts formed with cations such as sodium, potassium, ammonium, magnesium, calcium, polyamines such as spermine and spermidine, etc. (b) acid addition salts formed with inorganic acids, for example hydrochloric acid, hydrobromic acid, nitric acid, phosphoric acid, sulfuric acid and the like; (c) salts formed with organic acids such as, for example, acetic acid, alginic acid, ascorbic acid, benzoic acid, citric acid, fumaric acid, gluconic acid, maleic acid, methanesulfonic acid, naphthalenedisulfonic acid, naphthalenesulfonic acid, oxalic acid, palmitic acid, polyglutamic acid, p-toluenesulfonic acid, polygalacturonic acid, succinic acid, tartaric acid, tannic acid and the like; and (d) salts formed from elemental anions such as chlorine, bromine, and iodine.

In yet another embodiment, pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavouring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Generally, such carriers should be non-toxic to the recipient at the dosages and concentrations used. Ordinarily, the preparation of such compositions involves combining the therapeutic agent with one or more of the following: buffers, antioxidants, low molecular weight polypeptides, proteins, amino acids, carbohydrates including glucose, sucrose or dextrins, chelating agents such as EDTA, glutathione and other stabilizers and excipients. Neutral buffered saline or saline mixed with non-specific serum albumin are examples of suitable diluents

The pharmaceutical formulations of the present invention, which may conveniently be presented in unit dosage form, may be prepared according to conventional techniques well known in the pharmaceutical industry.

In yet another embodiment, the compositions of the present invention may be prepared and formulated as emulsions which are typically heterogeneous systems of one liquid dispersed in another in the form of droplets (Idson, 1988). Examples of naturally occurring emulsifiers used in emulsion formulations include acacia, beeswax, lanolin, lecithin and phosphatides. The application of emulsion formulations via dermatological, oral and parenteral routes and methods for their manufacture have been reviewed in the literature (Idson, 1988).

In one embodiment of the present invention, the compositions of oligonucleotides and nucleic acids can be formulated as microemulsions. A microemulsion is defined as a system of water, oil and amphiphile, which is a single optically isotropic and thermodynamically stable liquid solution (Rosoff, 1988).

Another embodiment of the present invention is the use of liposomes for the transfer and delivery of active ingredients to the site of action. Because the liposomal membrane is structurally similar to biological membranes, when liposomes are applied to a tissue, the liposomes start to merge with the cellular membranes. This fact has prompted extensive research in the use of liposomes as potential drug delivery modes.

In another embodiment, the use of penetration enhancers may be of use as a mode of drug delivery. Such agents are classified as belonging to one of five broad categories, i.e., surfactants, fatty acids, bile salts, chelating agents, and non-chelating non-surfactants (Lee et al., 1991).

In another related embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

The formulation of therapeutic compositions and their subsequent administration is believed to be within the skill of those in the art. Optimal dosing schedules can be calculated from measurements of drug accumulation in the body of the patient. Persons of ordinary skill can easily determine optimum dosages, dosing methodologies and repetition rates. Following successful treatment, it may be desirable to have the patient undergo maintenance therapy to prevent the recurrence of the disease state, wherein the oligonucleotide is administered in maintenance doses.

The present invention also relates to a recombinant nucleotide sequence comprising an antisense compound according to the invention. The recombinant nucleotide sequence can be inserted in an expression vector, such as a plasmid or virus or any other vector known to a person skilled in the art. Thus, the invention includes the antisense oligonucleotide sequences operably linked to one or more expression control elements, such that in vivo or in vitro expression of said antisense compound could be achieved. The vector capable of harbouring said antisense oligonucleotides can be of eukaryotic or prokaryotic origin.

One embodiment of the invention is a method of inhibiting the expression of NGAL in cells or tissues, wherein said cells or tissues is contacted in vivo or in vitro with the recombinant nucleotide sequence expressed by the recombinant vector. The invention also includes host cells transformed with these antisense oligonucleotide sequences operably linked to one or more expression control elements.

The present invention also provides transgenic cells as such, as well as transgenic non-human animals. Transgenic animals include animals comprising viable transgenic cells, or transgenic organs, as well as entire animals incorporating any one of the inventive antisense oligonucleotide sequences (SEQ ID NOs. 3 to 11) or functional parts thereof, in their genome under the control of a suitable expression cassette. Such animals are useful as research tools for investigations regarding the aetiology of NGAL related disorders, the progression, diagnosis and treatment of the same. As is well know in the art, these expression cassettes containing suitable promoters and enhancers are introduced into the cell of interest in the form of vectors such that expression of the desired antisense DNA sequence is achieved, resulting in an in vitro of in vivo inhibition of the production of NGAL. Thus, inventive sequences may thus be over-expressed, such that suppression of the intended target is achieved in the cells in which the antisense compound is expressed.

One embodiment of the present invention is thus such transgenic cells, organs or animals, and their use as models for investigating the nature and/or aetiology of NGAL related diseases, as models for evaluating the efficacy of pharmaceuticals against such diseases, as well as investigating the effect of known and suspected causative agents behind such diseases.

Having confirmed the involvement of NGAL in inflammatory disorders, the invention further provides screening assays for identifying an agent that modulates the activity of NGAL by altering the activation of the NGAL molecule

As the agent usable in the screening method of the invention, a newly synthesized compound, a commercial compound or a known compound which is registered in a chemical file but the various activities are unknown, a series of compounds obtained by the technology of combinatorial chemistry can be used. Also, a supernatant of culture of a microorganism, a natural component derived from a plant or a marine organism, an animal tissue extract, and the like can be used.

The method comprises contacting the NGAL, under conditions that allow an agent suspected of being able to alter the activity of NGAL to interact with NGAL such that a change in activity levels of NGAL can be easily seen Preferred mode of changes in the activity is the inhibition of NGAL activity or an agent with antagonistic effect.

According to the present invention, an assay for identifying an agent that alters the specific activity of NGAL can be, for example, an in vitro, or cell based assay and a suitable manner of monitory changes in NGAL activity.

In another embodiment, screening of compounds that have an antagonistic effect of NGAL can be done using solid phase combinatorial library approach.

The library can be, for example, be constructed as a one-bead-two-compounds library so that every bead contained a common quenched fluorogenic substrate and a different putative inhibitor. After incubation with NGAL, beads containing active inhibitors can be simply collected and the inhibitor compound structure analyzed using, for example a MALDI-TOF mass spectrometer (Franz et al., 2003).

While the present invention has been described with specificity in accordance with certain of its preferred embodiments, the following examples serve only to illustrate the invention and are not intended to limit the same. While they are typical of those that might be used, other procedures known to those skilled in the art may alternatively be adopted without resort to undue experimentation.

### EXAMPLES

### Example 1

### Identification of Human NGAL as Being Over-Expressed in Conditions of Inflammation

Collection of the Appropriate Biopsy Material

The biopsies were taken from patients who were selected on the basis of clinical and pathological evidence of having the inflammatory condition of CD or UC. A total of three biopsies were collected from a inflamed site in the colon, together with three biopsy samples from a non-inflamed region of a single individual patient. This was done for a total of 16 different patients of which eight were diagnosed having CD (patient 1-8) and eight having UC (patient 9-16). The UC patient group comprised 2 females and 6 males, the age range being 29-77 years. The CD age group correspondingly 3 females and 5 males, age range 27-59

The biopsies of each anatomical site of one patient were pooled and total RNA was isolated using Quiagen Rneasy Kit and a Pellet Pestel Motor Homogenizer according to the manufacturers protocol. A total of 32 samples of total RNA were isolated, two samples per patient: inflamed (target) and non-inflamed (control).

Performing cDNA Synthesis of the RNA

Two microgram of each RNA sample was used for a first strand cDNA synthesis using 10pM of the Oligo-dT-primer dT-joint (5′-TAG TCT ATG ATC GTC GAC GGC TGA TGA AGC GGC CGC TGG AGT TTT TTT TTT TTT TTT TTV-3′ (SEQ. ID. NO. 12) introducing to every synthesised cDNA molecule three restriction enzyme cutting sites: SalI, NotI and BpmI. The buffer, desoxynucleotide triphosphates (dATP, dCTP, dGTP and dTTP) and the enzyme reverse transcriptase (Superscript II) were purchased from Gibco BRL and the reactions were performed according to the manufactures guidelines. Briefly, the reaction mixture for first strand synthesis excluding the enzyme was preincubated for 5 min at 65° C. in a PCR machine (PCR sprint from Hybaid), chilled on ice, and then preheated to 42° C., before the enzyme Superscript II was added and incubated for 1 h at 42° C. in the PCR machine.

For the second strand synthesis, 41 μl second strand buffer mix were added to the reactions according to the provided protocol (Gibco BRL) and 4 μl E. coli Polymerase I (New England Biolabs), 1.5 μl E. coli DNA ligase (New England Biolabs) and 0.7 μl Rnase H (Gibco BRL) in a total volume of 160 μl. The reactions were incubated for 2.5 h at 16° C. in the PCR machine PCRsprint and then purified using the Quiagen PCR Purification Kit according to the protocol provided. Every sample was eluted with 32 μl of elution buffer and 26 μl of each sample was used for the following steps.

Amplification of the 3′-termini of the cDNAs

Due to limited amounts of material obtained from such biopsies, a pre-amplification step was necessary. For in vitro amplification of the 3′-end of cDNAs, 26 μl of cDNA from every sample was digested with 10 U of the restriction enzyme DpnII in a volume of 30 μl for 3h at 37° C. The cut cDNAs were purified once more using Quiagen PCR purification Kit and the cDNAs were eluted in 47 μl elution buffer. The following circular ligation step was performed in a volume of 50 μl including 44 μl of the DpnII cut cDNA and 2000 U T4 DNA ligase (New England Biolabs). These reaction mixtures were incubated at 22° C. for 1 h, heat inactivated by 65° C. for 10 min and 25 μl of each reaction mixture was used for the amplification step. A mixture for 5 reactions per sample was put together (5×50 μl=250 μl in total) containing 25 μl cDNA (DpnII cut and circular ligated), 25 μl 10× Advantage 2 PCR buffer (Clontech), 5 μl joint-Not primer (10 pmol/μl; 5′-TGA TGA AGC GGC CGC TGG-3′ (SEQ. ID. NO. 13.)), 5 μl joint-Sal primer (10pmol/μl; 5′-TTC ATC AGC CGT CGA CGA TC-3′ (SEQ. ID. NO. 14.), 5 μl 10 mM dNTP mix and 5 μl 50× Advantage 2 Taq-Polymerase (Clontech). For each sample the mix was distributed into 5 PCR reaction tubes and PCR performed under the following conditions: 1 min 94° C. then 16×(20 sec 94° C., 20 sec 55° C., 1 min 72° C.).

Four of the reactions per sample were removed and placed on ice and the optimal cycle number was determined with one of the reactions per sample. The optimal cycle number was determined to 18 cycles for all 32 samples, thus for the remaining four reactions per sample two additional cycles [2×(20 sec 94° C., 20 sec 55° C., 1 min 72° C.)] were performed. The 4 PCR reactions per sample were subsequently purified using the Quiagen PCR purification Kit. For the purification, the four reactions per sample were pooled (total of 200 μl) and then eluted with 34 μl elution buffer. The purified reactions were the starting material for the identification of the differentially expressed genes protocol.

Isolation of the Differentially Expressed cDNA (Subtraction Protocol) from Human Biopsies

Isolation of differentially expressed cDNAs was performed according to the protocol outlined in (von Stein O D, 2001) with minor modifications to the protocol.

Screening for the Differentially Expressed Genes

Upon construction of a cDNA library, 2.000 clones were plated out from each subtraction on one 22 cm² agar plate (. From these plates 384 colonies were picked and placed in 384 well plates with 70 μl LB medium/well (see Maniatis et al., 1989) (+ ampicillin 100 mg/ml) using BioPick machine of BioRobotics (Cambridge, UK). The bacterial clones were incubated over night at 37° C. and then used for colony PCR. This PCR was performed in 384 PCR well plates in a volume of 20 μl per sample. One PCR reaction included: 2 μl 10× PCR buffer, 0,4 μl Sport-Not primer (10 pmol 5′-CGT AAG CTT GGA TCC TCT AGA GC-3′ (SEQ ID NO 15), 0,4 μl of Sport-Sal primer (10 pmol 5′-TGC AGG TAC CGG TCC GGA ATT CC-3′ (SEQ ID NO 16)), 1,6 μl dNTP mix (25 mM each), 0,4 μl 0,1% Bromphenol blue and 0,5 μl DynAzyme Taq-polymerase (2 U/μl; Finnzyme). A master mix for all reactions was prepared, distributed and then inoculated with a 384 plastic replica. The PCR cycling parameters were: 2 min 94° C., 37 times (30 sec 94° C.; 30 sec 50° C., 1 min 72° C.) and 5 min 72° C.

Following amplification, PCR reactions were spotted on Hybond N⁺ membrane (Amersham) using Microgrid TAS of BioRobotics. All clones were spotted in duplicate and genomic DNA was used as guide dots. On one filter 383 genes of all four subtractions were positioned. 24 duplicates were made for analyses by hybridisation with different radioactive cDNA probes.

These filters were then hybridised with the radioactive labelled subtracted cDNAs of all eight patients. Sixteen filters were used in 16 different hybridisation experiments. For that 1 μl of the cDNAs were used for the labelling with Klenow polymerase. The hybridisation protocol was that of Church-protocol as outlined in (Church and Gilbert, 1984).

Phospho-imager Fujifilm BAS 1800II with BAS 1800 III R program, Array vision version 6.0 (Imaging Research Inc), sequencing, and BLAST analysis were used to determine the degree of differentially expression and the identity of the isolated differentially expressed genes.

Confirmation of True Differential Expression

To confirm results of the expression profiling experiment, RT-PCR analysis using gene-specific primers, and primers for alpha-actin (control) was performed. The original cDNAs derived from eight (8) individual UC patients and eight (8) individual CD patients were used. The cDNAs were diluted 1:250 in distilled water and a 5 μl aliquot used for one single PCR reaction. Reactions were performed in 50 μl 1× PCR buffer (provided with the Taq-polymerase; Finnzyme) total volume and included 0.5 μl 25 mM dNTP-mix, 10 pM forward and reverse primer of NGAL or alpha-actin and 1 unit of DynZyme (Taq-polymerase of Finnzyme). PCR reactions were performed in Thermohybaid Thermocycler under following conditions: 1 min 94° C. and N cycles (30 sec 94° C., 30 sec 55° C., 1 min 72° C.) and 5 min 72° C. For NGAL 30 cycles were performed (N=30), for alpha-actin 28 cycles (N=28). Upon completion, 5 μl of each reaction was loaded on a 1×TAE agarose gel and later stained with ethidium bromide.

| | | |
|---|---|---|
| NGAL forward: | 5′-GAG TTC ACG CTG GGC AAC ATT AAG-3′ | (SEQ.ID.NO.17) |
| NGAL reverse: | 5′-CAC TCA GCC GTC GAT ACA CTG GTC-3′ | (SEQ.ID.NO.18) |
| alpha-actin.forward: | 5′-GTG CAG GGT ATT AAC GTGTCA GGG-3′ | (SEQ.ID.NO.19) |
| alpha-actin.reverse: | 5′-CCA ACT CAA AGC AAG TAA CAG CCC ACG G-3′. | (SEQ.ID.NO.20) |

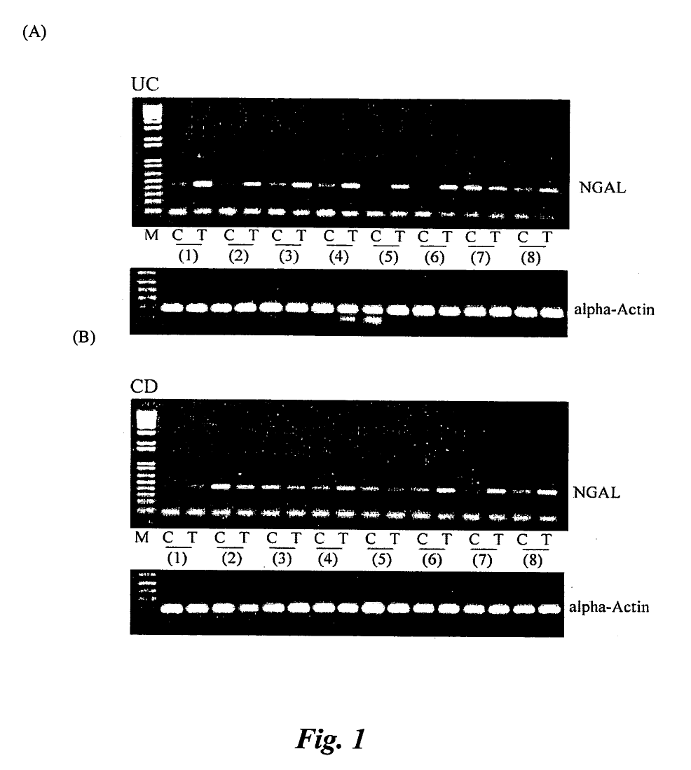

From these analysis it can be concluded that in both conditions of UC and CD, there is an up-regulation of NGAL in the majority of inflamed cases when compared to non inflamed matched control tissues. (see FIG. 1a and b and compare lines marked with (C) with the corresponding lane (T)). The actin control indicates that all samples had equal mRNA input and therefore increased expression seen in the target lanes (T) are true differences.

### Example 2

### Analysis of Antisense Oligonucleotide Inhibition of NGAL

Antisense modulation of NGAL expression can be assayed in a variety of ways well known in the art. For example, NGAL mRNA levels can be quantitated by, e.g., Northern blot analysis, competitive polymerase chain reaction (PCR), or real-time PCR (RT-PCR). RNA analysis can be performed on total cellular RNA or poly(A)+mRNA. Methods of RNA isolation are taught in, for example, Ausubel et al., 1993. Northern blot analysis is routine in the art and is taught in, for example, Ausubel et al., 1992. Real-time quantitative (PCR) can be conveniently accomplished using the commercially available ABI PRISM.TM. 7700 Sequence Detection System, available from PE-Applied Biosystems, Foster City, Calif., USA and used according to manufacturer's instructions. Other methods of PCR are also known in the art.

Like wise, NGAL protein levels can be quantitated in a variety of ways well known in the art, such as immunoprecipitation, Western blot analysis (immunoblotting), or ELISA. Antibodies directed to NGAL can be commercially acquired or one can generate an own antibody via conventional antibody generation methods. Methods for preparation of polyclonal antisera are taught in, for example, Ausubel et al., 1997. Preparation of monoclonal antibodies is taught in, for example, Ausubel et al., 1997.

Immunoprecipitation methods well known in the art can be found at, for example, Ausubel et al., 1998. Western blot (immunoblot) analysis is standard in the art and can be found at, for example, Ausubel et al., 1997. Enzyme-linked immunosorbent assays (ELISA) are standard in the art and can be found at, for example, Ausubel et al., 1991.

Suppression of Inflammation in a Colitis Mouse Model Using Antisense Oligonucleotides to NGAL

An animal model to induce inflammation in the large intestine of mice was introduced by Okayasu et al. in 1990. In this model oral dextran sulfate sodium (DSS) is utilized to induce inflammation (Axelsson, 1998). DSS can be given in the drinking water to mice and thereby induces a colitis resembling inflammatory bowel disease (IBD) in man. This inflammation inducing form of DSS has been shown to be optimal at a Mw of about 40-50 kD and with an approximate high content of up to 19% sulphur. It has been given to the animals at a concentration of about 2-5% (Okayasu 1990).

In this study we used DSS at a concentration of 2.5% dissolved in water, with a final pH 8.5 adjusted with NaOH. DSS was given orally to female SPF NMRI mice for 8 consecutive days to induce a stable colitis in all individuals. This type of experimentally induced colitis has been shown to be fully induced at day 4-5 after addition in the drinking water (Cooper et al., 1993).

The antisense substance as given by SEQ.ID.NO.3 was administered rectally to non-medicated or anaesthetized colitic animals. A shortened XRO feeding tube (Vygon, Ecouen, France) was inserted rectally, up to the level of the ligament of Treitz, and the substance, in a volume of 100 ul, was administered during slow retraction of the tubing with care, to avoid rectal leakage of substance. A single dose of 100 μg antisense in 100 μl water was administered. Therapeutic treatment was given once on day 8 while the DSS treatment continued another 10 days. On day 18 the animals were killed and submitted to analysis of clinical inflammatory parameters and histopathological examinations.

Clinical Signs

Each mouse was observed once daily during the study period. All signs of ill health and any behavioural changes were recorded. Animals showing severe signs of disease and losing more than 15% of its original body weight were killed.

Mortality and Necropsy

Mortality during the experimental period was recorded. At the end of the experimental period animals were killed by dislocation of the cervical spine. The abdomen was opened and the spleen was resected and weighed. The large intestine was excised from the ileocecal junction to the proximal rectum, close to its passage under the pelvisternum. The caecum was opened at the apex and faeces were carefully removed. The colon was opened longitudinally and the faeces were carefully removed with a spatula. Evaluation of colitis was made by recording clinical parameters such as mortality, colon length, spleen weight and diarrhoea, calculated as wet/dry weight of the faeces after drying 48 h at 60° C. The entire caecum and colon were fixed in 4% neutral buffered formaldehyde for microscopic examination.

Processing and Microscopic Examination

After fixation, the tissues sampled for microscopic examination were trimmed and specimens were taken from caecum and the mid portion of colon for histological processing. Additional specimens were taken when the first sample was difficult to interpret. The specimens were embedded in paraffin and cut at a nominal thickness of 5 μm, stained with haematoxylin and eosin, and examined under light microscope.

Verification of colitis and estimation of inflammation was performed by an experienced veterinary pathologist, having extensive experience of the histopathological evaluation of DSS-induced colitis in mice.

Diagnostic histopathology is based on a standardized grading system shown in Table 1.

TABLE 1
Histopathologic grading system
Colitis lesions:

| | |
|---|---|
| +/− | very mild (may be normal) (0) |
| + | mild (1) |
| ++ | moderate (2) |
| +++ | severe (3) |
| ++++ | very severe (4) |

Histological Analysis of Colonic Sections

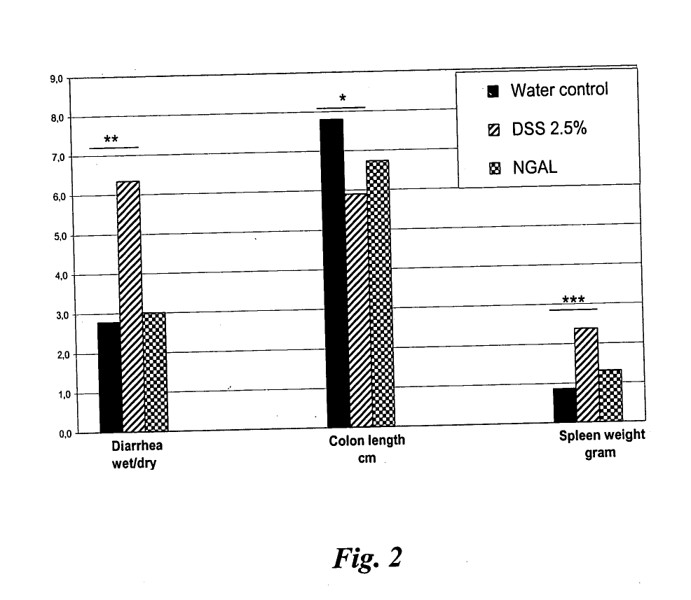

As outlined above, sections taken from the caecum and the mid portion of colon were used for histological processing. Staining was performed with haematoxylin and eosin. Sections were then examined by light microscopy and morphological changes noted. From FIG. 2 it can be concluded that a single rectal administration of antisense compound as given by SEQ.ID.NO 3, was sufficient to dramatically reduce the inflammation as seen on the commonly used physiological parameters monitored for signs of inflammatory improvements. There is a specific and statistical significant improvement in all measured parameters. That is to say, treated animals had less diarrhoea, had a more normal colon length, and had a more normal spleen weight

### Example 3

### In Vitro Screening of Human NGAL mRNA for Binding Site Accessible to Antisense Sequences

The effect of antisense compounds on target nucleic acid expression can be readily monitored in a variety of cell types provided that the target nucleic acid is present at measurable levels. To those skilled in the art, there are a number of well-established methods that can be employed to determine changes in levels of expressed target.

Treatment with Antisense Compounds

In order to identify an antisense compound that exhibits selective binding to human NGAL mRNA, and as a consequence causes a reduction in the amount of NGAL protein, a proprietary in vitro screening system was set up by the inventors. Within the coding region of SEQ. ID. NO. 1 (GenBank® Acc. No. BC033089), the inventors surprisingly identified a number of antisense sequences exhibiting very high inhibition.

The antisense sequences where then monitored for their ability to reduce the amount of human NGAL mRNA, by use of methods well known in the art, such as PCR or Northern blot analysis monitor the levels of target mRNA, whereas western blots indicate levels of protein encoded by the target mRNA. The potency of the antisense sequences was arbitrarily scored as a measure of degree of inhibition of the target sequence. Table 2 list the antisense sequences in groups in decreasing order of potency.

TABLE 2

The antisense sequences
grouped in their order of potency

| Sequence | |
|---|---|
| identification no. | Antisense sequence |
| SEQ.ID.NO.3 | 5′-CCC CAC ACC CAC ACC TGCT-3′ |
| SEQ.ID.NO.4 | 5′-GCC TGG GCA TGC AGA GCCC-3′ |
| SEQ.ID.NO.5 | 5′-CAG AGG TGG GGC TGG GATC-3 |
| SEQ.ID.NO.6 | 5′-CCC TGG AAT TGG TTG TCCT-3′ |
| SEQ.ID.NO.7 | 5′-TAG CTC TTG TCT TCT TTC A-3′ |
| SEQ.ID.NO.8 | 5′-TAG CAT GCT GGT TGT AGT T-3′ |
| SEQ.ID.NO.9 | 5′-GTA CTC CCT GTT TTG AGA A-3′ |
| SEQ.ID.NO.10 | 5′-CAG CTC CTT GGT TCT CCC G-3′ |
| SEQ.ID.NO.11 | 5′-TGC ATG GGT GGC ACT GTG G-3′ |

Antisense sequences SEQ ID NO.3-5 exhibited approximately 70-85% inhibition of NGAL mRNA levels relative to control. Antisense sequences SEQ ID NO. 6-7 exhibited approximately 55-75% inhibition of NGAL mRNA levels, relative to control. Antisense sequences SEQ ID NO. 8-11 exhibited approximately 35-55% inhibition of NGAL mRNA levels, relative to the control.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

### REFERENCES

Ausubel, F. M. et al., Current Protocols in Molecular Biology, Volume 2, pp. 11.2.1-11.2.22, John Wiley & Sons, Inc., 1991.

Ausubel F M, Brent R, Struhl K, Smith J A. Short Protocols in Molecular Biology: A Compendium of Methods from Current Protocols in Molecular Biology, John Wiley & Sons; 2nd edition, April 1992.

Ausubel, F. M. et al., Current Protocols in Molecular Biology, Volume 1, pp. 4.1.1-4.2.9 and 4.5.1-4.5.3, John Wiley & Sons, Inc., 1993.

Ausubel, F. M. et al., Current Protocols in Molecular Biology, Volume 2, pp. 11.12.1-11.12.9, John Wiley & Sons, Inc., 1997.

Ausubel, F. M. et al., Current Protocols in Molecular Biology, Volume 2, pp. 10.16.1-10.16.11, John Wiley & Sons, Inc., 1998.

Axelsson L-G, Landstrom E, Bylund-Fellenius A-C. Experimental colitis induced by dextran sulphate sodium in mice: beneficial effects of sulfasalazine and olsalazine. Aliment Pharmacol Therap 1998;12:925-934.

Betsuyaku T, Nishimura M, Takeyabu K, Tanino M, Venge P, Xu S, Kawakami Y. Neutrophil granule proteins in bronchoalveolar lavage fluid from subjects with subclinical emphysema. Am J Respir Crit Care Med 1999 June;159(6):1985-91.

Carlson M, Raab Y, Seveus L, Xu S, Hallgren R, Venge P. Human neutrophil lipocalin is a unique marker of neutrophil inflammation in ulcerative colitis and proctitis. Gut 2002 April;50(4):501-6.

Church G M, Gilbert W. Genomic sequencing. Proc Natl Acad Sci USA. 1984;81:1991-5.

Cooper H S, Murthy S N, Shah R S, Sedergran D J. Clinicopathologic study of dextran sulfate sodium experimental murine colitis. Lab Invest 1993 August;69(2):238-49.

Eichler I, Nilsson M, Rath R, Enander I, Venge P, Koller D Y. Human neutrophil lipocalin, a highly specific marker for acute exacerbation in cystic fibrosis. Eur Respir J 1999 November;14(5):1145-9.

Franz A H, Liu R, Song A, Lam K S, Lebrilla C B. High-throughput one-bead-one-compound approach to peptide-encoded combinatorial libraries: MALDI-MS analysis of single TentaGel beads. J Comb Chem. 2003 Mar-Apr;5(2):125-37

Idson, in Pharmaceutical Dosage Forms, Lieberman, Rieger and Banker (Eds.), 1988, Marcel Dekker, Inc., New York, N.Y., volume 1, p. 199).

Jensen S, Gassama M P, Heidmann T. Taming of transposable elements by homology-dependent gene silencing. Nat Genet 1999 February;21(2):209-12

Ketting R F, Haverkamp T H, van Luenen H G, Plasterk R H. Mut-7 of C. elegans, required for transposon silencing and RNA interference, is a homolog of Werner syndrome helicase and RNaseD. Cell 1999 Oct. 15;99(2): 133.41

Kjeldsen L, Johnsen A H, Sengelov H, Borregaard N. Isolation and primary structure of NGAL, a novel protein associated with human neutrophil gelatinase. J Biol Chem 1993 May 15;268(14):10425-32.

Lee V H, Yamamoto A, Kompella U B. Mucosal penetration enhancers for facilitation of peptide and protein drug absorption. Crit Rev Ther Drug Carrier Syst 1991;8(2):91-192.

Lucy A P, Guo H S, Li W X, Ding S W. Suppression of post-transcriptional gene silencing by a plant viral protein localized in the nucleus. EMBO J 2000 Apr. 3;19(7): 1672-80.

Mallbris L, O'Brien K P, Hulthen A, Sandstedt B, Cowland J B, Borregaard N, Stahle-Backdahl M. Neutrophil gelatinase-associated lipocalin is a marker for dysregulated keratinocyte differentiation in human skin. Exp Dermatol 2002 December;11(6):584-91.

Maniatis T, Sambrook J, Fritsch E F. Molecular Cloning, A laboratory Manual, Appendix A.1 Cold Spring Habor Laboratory; 2^{nd} Ed (1989).

Nielsen B S, Borregaard N, Bundgaard J R, Timshel S, Sehested M, Kjeldsen L. Induction of NGAL synthesis in epithelial cells of human colorectal neoplasia and inflammatory bowel diseases. Gut 1996 March;38(3):414-20.

Okayasu I, Hatakeyama S, Yamada M, Ohkusa T, Inagaki Y, Nakaya R.A novel method in the induction of reliable experimental acute and chronic ulcerative colitis in mice. Gastroenterology 1990 March;98(3):694-702.

Ratcliff F G, MacFarlane SA, Baulcombe D C. Gene silencing without DNA. ma-mediated cross-protection between viruses Plant Cell 1999 July;11(7): 1207-16.

Rosoff, in Pharmaceutical Dosage Forms, Lieberman, Rieger and Banker (Eds.). 1988, Marcel Dekker, Inc., New York, N.Y., volume 1, p. 245.

Shipley J M, Wesselschmidt R L, Kobayashi D K, Ley T J, Shapiro S D. Metalloelastase is required for macrophage-mediated proteolysis and matrix invasion in mice. Proc Natl Acad Sci USA 1996 Apr. 30;93(9):3942-6.

Tabara H, Sarkissian M, Kelly W G, Fleenor J, Grishok A, Timmons L, Fire A, Mello C C. The rde-1 gene, RNA interference, and transposon silencing in C. elegans. Cell 1999 Oct. 15;99(2): 123-32.

Tschesche H, Zolzer V, Triebel S, Bartsch S. The human neutrophil lipocalin supports the allosteric activation of matrix metalloproteinases. Eur J Biochem 2001 April;268(7):1918-28.

von Stein O D. Isolation of differentially expressed genes through subtractive suppression hybridization. Methods Mol Biol 2001;175:263-78.

Yan L, Borregaard N, Kjeldsen L, Moses M A. The high molecular weight urinary matrix metalloproteinase (MMP) activity is a complex of gelatinase B/MMP-9 and neutrophil gelatinase-associated lipocalin (NGAL). Modulation of MMP-9 activity by NGAL. J Biol Chem 2001 Oct. 5;276(40):37258-65.

1. A compound 8 to 50 nucleobases in length targeted to a nucleic acid molecule encoding neutrophil gelatinase associated lipocalin (NGAL), characterized in that said compound specifically hybridizes to and inhibits the translation of NGAL.

2. A compound according to claim 1, wherein the target sequence is SEQ ID NO. 1 or equivalent functional homologues thereof.

3. The compound according to claim 1, wherein said compound is an antisense oligonucleotide complementary to the mRNA.

4. The compound according to claim 1, wherein the oligonucleotide is a DNA molecule.

5. The compound according to claim 1, wherein the oligonucleotide is a RNA molecule.

6. The compound according to claim 3, wherein the antisense oligonucleotide has a sequence selected from the group consisting of SEQ ID NO. 3-11.

7. The compound according to claim 3, wherein the oligonucleotide is RNAi comprising at least an 8 nucleotide portion of a sequence selected from the group of SEQ.ID.NO 3-11 and having a total length of no more than 25 nucleotides.

8. A compound 8 to 50 nucleobases in length targeted to a nucleic acid molecule encoding NGAL, said compound being an antisense oligonucleotide which specifically hybridizes to and inhibits the translation of NGAL in a human subject, characterized in that the compound is chemically modified by substitution in a non-bridging oxygen atom of the antisense nucleic acid backbone with a moiety selected from the group consisting of methane phosphate, methyl phosphate, and phosphorothioate.

9. The compound according to claim 8, wherein the substitution occurs at one or more nucleotides selected from the 3′ end or the 5′ end or both.

10. The compound according to claim 8, wherein the substitution occurs at one or more nucleotides at any position along the entire length of said oligonucleotide.

11. The compound according to any one of the preceding claims, wherein said compound is an antisense oligonucleotide composed of DNA or RNA or an analogue or mimic of DNA or RNA including but not restricted to the following: methylphosphonate, N3′->P5′-phosphoramidate, morpholino, peptide nucleic acid (PNA), locked nucleic acid (LNA), arabinosyl nucleic acid (ANA), fluoro-arabinosyl nucleic acid (FANA) methoxy-ethyl nucleic acid (MOE).

12. The compound according to claim 1, wherein said compound is an antisense oligonucleotide that is a homo or heteropolymer containing combinations of the above DNA or RNA or analogues or mimics of DNA or RNA.

13. The compound according to any one of claims 2-7, wherein in that said oligonucleotide comprises at least one modified sugar moiety nucleobase.

14. The compound of claim 13, wherein the modified sugar moiety is a 2′-O-methoxyethyl sugar moiety.

15. A composition comprising the compound according to any one of the preceding claims and a pharmaceutically acceptable carrier or diluent.

16. The composition of claim 15, wherein said composition further comprises a colloidal dispersion system.

17. A method of inhibiting the translation of NGAL in cells or tissues, characterized in that said cells or tissues are contacted with the compound of any one of claims 1-14 thereby inhibiting the translation of NGAL.

18. A method of inhibiting the translation of NGAL in cells or tissues, characterized in that said cells or tissues are contacted with the composition of any one of claims 15-16 thereby inhibiting the translation of NGAL.

19. The method according to claims 17 or 18, wherein the inhibition of the NGAL expression suppresses a NGAL dependent process in a human subject.

20. The method according to claim 19, wherein the NGAL dependent process is one of inflammatory bowel disease, such as ulcerative colitis and Crohn's disease, rheumatoid arthritis, psoriasis, and asthma.

21. A method of preventing, alleviating or treating a NGAL dependent disorder in a human patient, characterized in that NGAL expression is suppressed in one or more cells in said patient.

22. The method according to claim 21, wherein said NGAL dependent disorder is one of inflammatory bowel disease, such as ulcerative colitis and Crohn's disease, rheumatoid arthritis, psoriasis, and asthma.

23. A recombinant nucleotide sequence comprising a compound according to any one of claims 1-14.

24. A recombinant expression vector comprising the recombinant nucleotide sequence according to claim 23.

25. The recombinant expression vector according to claim 24, wherein the vector is of eukaryotic or prokaryotic origin.

26. A method of inhibiting the expression of NGAL in cells or tissues, characterized in that said cells or tissues are contacted in vivo or in vitro with the recombinant nucleotide sequence expressed by the recombinant vector according to claim 25.

27. A recombinant host cell produced by the method of claim 26.

28. A transgenic non-human animal, wherein said animal carries at least one sequence according to claim 6 functionally inserted in at least one cell.

29. The transgenic animal according to claim 28, wherein the at least one functionally inserted sequence is over-expressed.

30. A method of inhibiting the expression of NGAL in cells or tissues, wherein a composition according to claim 15 or 16 is administered to a human in a therapeutically effective dose with a pharmaceutically acceptable carrier.

31. A method of diagnosing inflammatory bowel disease in a human subject, characterized in the method comprising the step of screening for the presence or absence of the expression of NGAL, wherein the expression of NGAL is taken as an indication of inflammatory bowel disease.

32. A compound 8 to 50 nucleobases in length targeted to a nucleic acid molecule encoding 24p3/uterocalin, characterized in that said compound specifically hybridizes to and inhibits the translation of 24p3/uterocalin.

33. A compound according to claim 32, wherein the target sequence is SEQ ID NO. 2 or equivalent functional homologues thereof.

34. The compound according to claim 33, wherein said compound is an antisense oligonucleotide complementary to the mRNA.

35. The compound according to claim 33, wherein the oligonucleotide is a DNA molecule.

36. The compound according to claim 33, wherein the oligonucleotide is a RNA molecule.

37. The compound according to claim 33, wherein the antisense oligonucleotide has a sequence selected from the group consisting of SEQ ID NO. 3-11.

38. A transgenic non-human animal, wherein said animal carries at least one sequence according to claim 30 functionally inserted in at least one cell.

39. The transgenic animal according to claim 38, wherein the at least one functionally inserted sequence is over-expressed.


SEQUENCE LISTING (20 sequences)

SEQ ID NO: 1 - DNA, 822 bp, Homo sapiens
  human NGAL; Genbank Acc. No. BC033089
tcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaaaaaaaaaaaaaaaaaa

SEQ ID NO: 2 - DNA, 911 bp, Mus musculus
  mouse lipocalin 2; Genbank Acc. No. XM_130171
taaaaagcccgctggggagtcctcctcactctctgctcttcctcctccagcacacatcagacctagtagctgtggaaaccatggccctgagtgtcatgtgtctgggccttgccctgcttggggtcctgcagagccaggcccaggactcaactcagaacttgatccctgccccatctctgctcactgtccccctgcagccagacttccggagcgatcagttccggggcaggtggtacgttgtgggcctggcaggcaatgcggtccagaaaaaaacagaaggcagctttacgatgtacagcaccatctatgagctacaagagaacaatagctacaatgtcacctccatcctggtcagggaccaggaccagggctgtcgctactggatcagaacatttgttccaagctccagggctggccagttcactctgggaaatatgcacaggtatcctcaggtacagagctacaatgtgcaagtggccaccacggactacaaccagttcgccatggtatttttccgaaagacttctgaaaacaagcaatacttcaaaattaccctgtatggaagaaccaaggagctgtcccctgaactgaaggaacgtttcacccgctttgccaagtctctgggcctcaaggacgacaacatcatcttctctgtccccaccgaccaatgcattgacaactgaatgggtggtgagtgtggctgactgggatgcgcagagacccaatggttcaggcgctgcctgtctgtctgccactccatctttcctgttgccagagagccacctggctgccccaccagccaccataccaaggagcatctggagcctcttcttatttggccagcactccccatccacctgtcttaacaccaccaatggcgtcccctttctgctgaataaatacatgccccc

SEQ ID NO: 3 - DNA, 19 bp, Artificial Sequence
  Description of Artificial Sequence Synthetic antisense oligonucleotide
ccccacacccacacctgct

SEQ ID NO: 4 - DNA, 19 bp, Artificial Sequence
  Description of Artificial Sequence Synthetic antisense oligonucleotide
gcctgggcatgcagagccc

SEQ ID NO: 5 - DNA, 19 bp, Artificial Sequence
  Description of Artificial Sequence Synthetic antisense oligonucleotide
cagaggtggggctgggatc

SEQ ID NO: 6 - DNA, 19 bp, Artificial Sequence
  Description of Artificial Sequence Synthetic antisense oligonucleotide
ccctggaattggttgtcct

SEQ ID NO: 7 - DNA, 19 bp, Artificial Sequence
  Description of Artificial Sequence Synthetic antisense oligonucleotide
tagctcttgtcttctttca

SEQ ID NO: 8 - DNA, 19 bp, Artificial Sequence
  Description of Artificial Sequence Synthetic antisense oligonucleotide
tagcatgctggttgtagtt

SEQ ID NO: 9 - DNA, 19 bp, Artificial Sequence
  Description of Artificial Sequence Synthetic antisense oligonucleotide
gtactccctgttttgagaa

SEQ ID NO: 10 - DNA, 19 bp, Artificial Sequence
  Description of Artificial Sequence Synthetic antisense oligonucleotide
cagctccttggttctcccg

SEQ ID NO: 11 - DNA, 19 bp, Artificial Sequence
  Description of Artificial Sequence Synthetic antisense oligonucleotide
tgcatgggtggcactgtgg

SEQ ID NO: 12 - DNA, 60 bp, Artificial Sequence
  Description of Artificial Sequence Primer
tagtctatgatcgtcgacggctgatgaagcggccgctggagttttttttttttttttttv

SEQ ID NO: 13 - DNA, 18 bp, Artificial Sequence
  Description of Artificial Sequence Primer
tgatgaagcggccgctgg

SEQ ID NO: 14 - DNA, 20 bp, Artificial Sequence
  Description of Artificial Sequence Primer
ttcatcagccgtcgacgatc

SEQ ID NO: 15 - DNA, 23 bp, Artificial Sequence
  Description of Artificial Sequence Primer
cgtaagcttggatcctctagagc

SEQ ID NO: 16 - DNA, 23 bp, Artificial Sequence
  Description of Artificial Sequence Primer
tgcaggtaccggtccggaattcc

SEQ ID NO: 17 - DNA, 24 bp, Artificial Sequence
  Description of Artificial Sequence Primer
gagttcacgctgggcaacattaag

SEQ ID NO: 18 - DNA, 24 bp, Artificial Sequence
  Description of Artificial Sequence Primer
cactcagccgtcgatacactggtc

SEQ ID NO: 19 - DNA, 24 bp, Artificial Sequence
  Description of Artificial Sequence Primer
gtgcagggtattaacgtgtcaggg

SEQ ID NO: 20 - DNA, 28 bp, Artificial Sequence
  Description of Artificial Sequence Primer
ccaactcaaagcaagtaacagcccacgg